(19) United States
(12) Patent Application Publication
(10) Pub. No.: US 2004/0014681 A1
(43) Pub. Date: Jan. 22, 2004

(54) Method for treating dermatoses and tissue damage

(76) Inventor: Ralph Ryback, Bethesda, MD (US)

Correspondence Address: CONNOLLY BOVE LODGE & HUTZ LLP, SUITE 800, 1990 M STREET NW, WASHINGTON, DC, 20036-3425, US

(21) Appl. No.: 10/437,433

(22) Filed: May 14, 2003

Related U.S. Application Data
(60) Provisional application No. 60/379,747, filed on May 14, 2002.

(51) Int. Cl. A61K 31/7008; A61K 31/343; A61K 31/255; A61K 31/36

(52) U.S. Cl. 514/23; 514/464; 514/517

(57) ABSTRACT

Dermatoses and/or tissue damage are treated by administering a compound of the following formula (I)

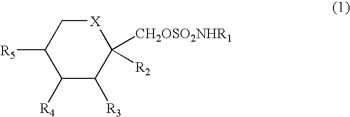

### TECHNICAL FIELD

The present invention relates to a method for treating dermatoses and tissue damage. According to the present invention, various sulfamates including topiramate are used for treating dermatoses and tissue damage.

### BACKGROUND OF INVENTION

Compounds of Formula(1):

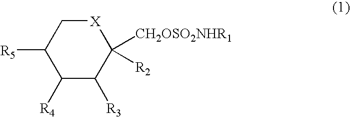

are known antiepileptic compounds that are highly effective anticonvulsants in animal tests (Maryannoff et al. J. Med Chem. 1987, 30, 880-887; Maryannoff et al., Bioorg. Med. Chem. Lett. 1993, 3, 2653-2656; Shank et al., Epilepsia 1994, 35, 450-460; Maryannoff et al., J. Med. Chem. 1998 41, 1315-1343). These compounds are described for example in U.S. Pat. No. 4,513,006, No. 5,242,942, and No. 5,384,327.

One of these compounds 2,3:4,5-bis-O-(1-methylethylidence)-β-D-fructopyranose sulfamate known as topiramate has been demonstrated in clinical trials of human epilepsy to be effective as adjunctive therapy or as monotherapy in treating simple and complex partial seizures and secondarily generalized seizures (Faught et al., Epilepsia 1995, 36 (Sr), 33; S. K. Sachdeo et al. Epilepsia 1995, 36 (S4), 33; GLAUSER, Epilepsia 1999, 40 (S5), S71-80; SACHDEO, Clin. Pharmacokiet 1998, 34, 335-346), and is currently marketed for the treatment of seizures in patients with simple and complex partial epilepsy and seizures in patients with primary or secondary generalized seizures in the United States, Europe and many other markets throughout the world.

Compounds of Formula (1) were initially found to possess anticonvulsant activity in the traditional maximal electroshock seizure (MES) test in mice (Shank et al., Epilepsia 1994, 35, 450-460). Subsequent studies revealed that Compounds of Formula 1 were also highly effective in the MES test in rats. Topiramate was also found to effectively block seizures in several rodent models of epilepsy (Nakamura et al., Eur. J. Pharmacol. 1994, 254, 83-89), and in an animal model of kindled epilepsy (Wauquier et al., Epilepsy Res. 1996, 24, 73-77)

Currently topical glucocorticoids are the most frequently used drugs in treating dermatoses and tissue damage including dermatitis. Attempts have been made to optimize potency, especially the immuno-suppressive and anti-inflammatory properties while minimizing adverse side effects. These immuno-suppressive and anti-inflammatory properties have a broad range of application outside of direct skin topical treatments which will be discuss later as it applies to the present invention.

Side effects from the use of topical steroids have become more prevalent since the introduction of the higher potency topical steroids. Using these products on thin or denuded skin, on the elderly or pediatric population, or under occlusion increases the incidence of side effects. Striae and atrophy, the most commonly observed side effects, occur with prolonged use and are more likely to occur in areas of sweating, occlusion, or high penetration such as the axilla or groin. In general, atrophy does not occur until the agent has been used for 3 to 4 weeks and is usually reversible. Striae, which develop when the weakened skin is stretched, are not reversible. Prolonged treatment can also result in “steroid acne,” which is characterized by crops of dense, inflamed pustules in the same developmental stage. These lesions occur on the face, chest and back. Perioral and periocular dermatitis have been associated with the use of topical steroids and usually improve with the cessation of the steroid.

Topical steroids can also cause suppression of the pituitary-adrenal axis. Growth retardation and iatrogenic Cushing's syndrome are known but rare complications of topical steroid therapy.

The following table illustrates relative responsiveness to topically applied corticosteroids

TABLE 1
Responsiveness of Dermatoses and Tissue Damage to Topical Application
of Corticosteroids

| HIGHLY | MODERATELY | LEAST |
|---|---|---|
| RESPONSEIVE | RESPONSIVE | RESPONSIVE |
| Psoriasis | Psoriasis | Palmoplantar |
| (intertriginous) | Atopic dermatitis | psoriasis |
| Atopic dermatitis | (adults) | Psoriasis of nails |
| (children) | Nummular eczema | Dyschidrotic eczema |
| Seborrheic dermatitis | Primary irritant | Lupus erythematosus |
| Intertrigo | dermatitis | Pemphigus |
| | Papular Urticaria | Lichen planus |
| | Parapsoriasis | Granuloma annulate |
| | Lichen simplex | Necrobiosis |
| | chronicus | lipodicadiabeticorum |
| | | Sarcoidosis |
| | | Allergic contact |
| | | dermatitis, acute |
| | | phase |
| | | Insect bites |

Other topical treatments such as coal tar, and keratolytics can be cumbersome, and have limited efficacy. However, since many of the currently available systemic treatments are associated with unacceptable toxicity or side effects, there is the need for additional treatment options.

### SUMMARY OF INVENTION

It has been found according to the present invention that compounds of the formula I below are useful in treating dermatoses and tissue damage responsive to corticosteroid as noted above (table 1), as well as, contact dermatitis, vitiligo, sunburn, burns, self-induced skin lesions and tissue damage, photosensitive dermatoses, and generalized, winter, senile and essential pruritis. This invention is also useful in treating the broad range of inflammatory responses and improving healing in tissue damage by direct application to involved tissues as well as in cases involving grafts and transplants including mechanical devices. Compounds employed according to the present invention have the following formula (I)

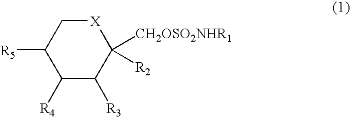

wherein

X is CH₂ or oxygen;

R₁ is hydrogen or alkyl and

R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II):

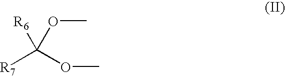

wherein

R₆ and R₇ are the same or different and are hydrogen, lower alkyl or are alkyl and are joined to form a cyclopentyl or cyclohexyl ring.

One aspect of the present invention relate to the typical application of a compound of formula (1) as defined above, preferably topiramate to treat a patient suffering from dermatoses and tissue damage. The compositions employed for the topical application include aerosols and foams, medicated tape and skin patches, pastes, oils, tinctures, aqueous solutions, creams and ointments.

It has been observed according to the present invention that treatment with a compound of the above formula (1) can be employed for treating all dermatoses and tissue damage responsive to the topical application of corticosteroids. However, this does not exclude types of tissue damage where corticosteroids would not be indicated but where this invention would be beneficial.

Accordingly, another aspect of the present invention relates to treating a patient suffering from dermatoses and/or tissue damage other than psoriasis by administering an effective amount of a compound of the above formula (1) by any mode of administration.

### BEST AND VARIOUS MODES FOR CARRYING OUT INVENTION

According to the present invention, various sulfamates have been found to be useful for treating dematoses and tissue damage diseases. The sulfamates employed pursuant to the present invention have the following formula (I):

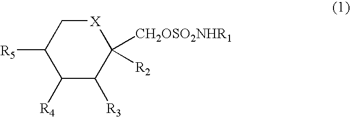

wherein

X is CH₂ or oxygen;

R₁ is hydrogen or alkyl and

R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II):

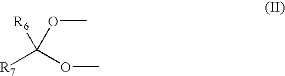

R₆ and R₇ are the same or different and are hydrogen, lower alkyl or are alkyl and are joined to form a cyclopentyl or cyclohexyl ring.

R₁ in particular is hydrogen or alkyl of about 1 to 4 carbons, such as methyl, ethyl and iso-propyl. Alkyl throughout this specification includes straight and branched chain alky. Alkyl groups for R₂, R₃, R_{4,} R₅, R₆ and R₇ are of about 1 to 3 carbons and include methyl, ethyl, iso-propyl and n-propyl. When X is CH₂, R₄ and R₅ may combine to form a benzene ring fused to the 6-membered X-containing ring, i.e., R⁴ and R⁵ are defined by the alkatrienyl group ═C—CH═CH—CH═.

A particular group of compounds of formula (I) is that wherein X is oxygen and both R₂ and R₃ and R₄ and R₅ together are methylenedioxy groups of the formula (II), wherein R₆ and R₇ are both alkyl such as methyl. A second group of compounds is that wherein X is CH₂ and R₄ and R₅ are joined to form a benzene ring. A third group of compounds of formula (I) is that wherein both R₂ and R₃ are hydrogen.

The preferred compound employed pursuant to the present invention is topiramate.

The compounds of formula (I) may be synthesized by the following methods:

(a) Reaction of an alcohol of the formula RCH₂OH with a chlorosulfamate of the formula ClSO₂NH₂ or ClSO₂NHR₁ in the presence of a base such as potassium t-butoxide or sodium hydride at a temperature of about −20 to 25 C and in a solvent such as toluene, THF, or dimethylformamide wherein R is a moiety of the following formula (III):

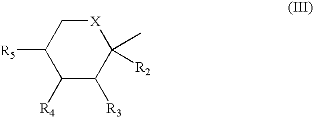

(b) Reaction of an alcohol of the formula RCH₂OH with a sulfurylchloride of the formula SO₂Cl₂ in the presence of a base such as triethylamine or pyridine at a temperature of about −40 to 25 C in a solvent such as diethyl ether or methylene chloride to produce a chlorosulfate of the formula RCH₂OSO₂Cl may then be reacted with an amine of the formula R¹NH₂ at a temperature of about 40 to 25 C in a solvent such as methylene chloride or acetonitrile to produce a compound of formula (I). The reaction conditions for (b) are also described by Tsuchiya, et al. in Tetrahedron Lett., 1978, 3365

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by Hedayatullah in Tetrahedron Lett. 1975, 2455. The azidosulfate is then reduced to a compound of formula (I) wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula (II) may be obtained by the method of Brady in Carbohydr. Res. 1970, 14, 35 or by reaction of the trimethylsilyl enol ether of a R₆COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by Larson, et al. in J. Org. Chem. 1973, 38 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such as diglyme, THF or toluene at a temperature of about 0 to 100° C., e.g. as described by H. O. House in “Modern Synthetic Reactions”, 2^{nd} Ed., pages 45 to 144 (1972).

The compounds of formula I: may also be made by the process disclosed U.S. Pat. No. 4,513,006, No. 5,242,942, No. 5,384,327 and No. 5,760,006 which are incorporated by reference herein.

The compounds of formula I include the various individual isomers as well as the racemates thereof, e.g., the various alpha and beta attachments, i.e., below and above the plane of the drawing, of R², R³, R⁴ and R⁵ on the 6-membered ring. More particularly, the oxygen of the methylenedioxy group (II) is attached on the same side of the 6-membered ring.

The conditions treated topically according to the present invention include those dermatitis conditions that have shown responsiveness to the topical application of corticosteroids (table 1) noted previously, as well as, contact dermatitis such as poison ivy and poison oak; atopic dermatitis , vitiligo, eczema, psoriasis, skin burn, photosensitive dermatitis, generalized, winter, senile and essential pruritis, and other types of tissue damage.

In another embodiment of the present invention is a method for treating seborrheic dermatitis with the mildest form known as “dandruff” or seborrhea which is also common in infants and is known as “cradle cap”, eczema, urticaria, eczematous dermatitis, contact dermatitis, localized neurodermatitis, seborrheic dermatitis, exfoliative dermatitis, pityriasis rosea, drug eruptions, stasis dermatitis or varicose eczema, erythema multiforme, alopecia areata, scarring alopecia (e.g. caused by lichen planus, cutaneous lupus, linear scleroderma, discoid lupus, sarcoidosis), vitiligo, and telangiectasis (e.g. caused by dermatomyositis, lupus erythematosus, and scleroderma).

In yet another embodiment of the present invention is a method for treating primary blistering diseases including pamphigus, bullous pemphigoid, herpes gestationis, cicatricial pemphigoid, dermatitis herpetiformis, linear IgA disease, and epidermolysis bullosa acquisita; secondary blistering diseases including contact dermatitis, both allergic and irritant forms, phototoxic eruptions resembling exaggerated sunburn in sun-exposed areas, associated with a variety of drugs( including but not limited to thiazides, deoxycycline, sulfonamides, penicillin, NSAIDs), burns including sunburn, and toxic epidermal necrolysis.

In another embodiment of this present invention is a method for treating by direct application and/or coating orchitis/vasectomy reversal procedures, allergic/atopic diseases, eczema, allergic contact dermatitis, allergic conjunctivitis, transplants, organ transplant rejection, graft-versus-host disease, trauma/hemorrhage, burns, ionizing radiation exposure, chronic inflammatory pathologies, atopic diseases, hypersensitivity reactions, conjunctivitis, urticaria, dermatitis, graft rejection of any organ or tissue, kidney transplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions.

The compositions employed for topical application can be in the form of aerosols, foams, medicated tape, skin patches, pastes, oils, tinctures, aqueous solutions, creams, and ointments. It has been found that the compositions typically have a pH of about 7 to about 10, preferably about 7 to about 8.5 and most preferably about 7 to about 8. If the pH value is lower than about 7, the compositions do not exhibit adequate stability. In addition, the higher the pH, the longer the observed clinical effectiveness of the preparation (and the inferred chemical stability of topiramate or similar active component) and hence longer effective shelf life. However, pH values above about 8 are not the preferred ones since the compositions tend to be somewhat irritating at the higher pH values.

In the case of a cream or ointment the topical compositions, according to the present invention, typically contain about 0.001 mg to about 3.0 mg of topiramate per milliliter of cream or ointment, and more typically 0.2 to 0.6 mg/ml.

For purposes of illustration and as examples, creams and ointments will be discussed in this context. However, for example aqueous, saline, or shampoo like solutions may be superior for certain applications.

Water-washable cream bases: These bases are pleasant for the patient to use, are non-greasy, and are almost always indicated when treating intertriginous and hairy areas. Their disadvantage is that they can be too drying. A number of medications, as specifically indicated, can be added to these bases (i.e., menthol, sulfur, tars, hydrocortisone, triamcinolone, antibiotics).

a. Unibase

b. Vanicram

c. Acid Mantle Creme

d. Dermovan

e. Unscented cold cream (not water-washable)

Ointment bases: These Vaseline-type bases are, and should be, the most useful in dermatology. Although not as pleasant for the patient to use as the cream bases, their greasy quality alleviates dryness, removes scales, and enables the medicaments to penetrate the skin lesions.

a. White petrolatum (USP)

b. Zinc oxide ointment (USP)

c. Aquaphor (contains lanolin)

d. Eucerin (contains lanolin)

The pH between 7 and 10 can be achieved by adding alkalinizing or antacid agents. Sodium bicarbonate, sodium carbonate, sodium citrate, potassium citrate, citric acid, tricitrates, and other citrate salt preparations, sodium lactate tromethamine aluminum hydroxide and aluminum salts (e.g., carbomate, phosphate, dihydroxyaluminum-sodium carbonate, calcium carbonate, magnesium containing salts (e.g. carbonate, hydroxide, oxide, trisilicate), dihydroxyaluminum, and magaldrate are some examples of alkalinizing or antacid agents which may be used in stabilizing the compositions of the present invention at a pH between 7 and 10. These cream or ointment bases and other preparations can also be refrigerated to maintain the ideal pH and to extend their effective shelf life.

In addition, according to the present invention, dermatoses other than psoriasis is treated by modes of administration in addition to topical administration.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

For pharmaceutical administration, one or more of the compounds of formula (I) may be administered by any suitable means, as would be apparent to one skilled in the art. More particularly, the compound(s) of formula (I) may be administered by any parenteral method, including, but not limited to oral, pulmonary, intraperitoneal (ip), intravenous (iv), intramuscular (im), subcutaneous (sc), transdermal, buccal, nasal, sublingual, ocular, rectal and vaginal. It will be readily apparent to those skilled in the art that any dose or frequency of administration that provides the therapeutic effect described herein is suitable for use in the present invention. A preferred mode of administration other than topical is oral. Oral administration is especially advantageous when treating patients suffering from a dermatoses that exists in a relatively wide spread area on a patients body. In fact, in some cases such as a wide spread dermatitis it can be advantageous to employ a combination of modes of administration such as topical along with oral. When employing oral administration, the dosage is typically about 10 to about 200 mg/day and more typically about 25 to about 75 mg/day depending upon the weight of the patient.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this invention can be administered by any conventional method available for use in conjunction with pharmaceuticals, either as individual therapeutic agents or in a combination of therapeutic agents.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired.

Dosage forms (compositions suitable for administration) typically contain about 0.01 mg to about 50 mg of active ingredient per unit.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parental administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethlcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alklypyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa.

Moreover, the compounds of the present invention can be administered in the form of nose drops, or metered dose and a nasal or buccal inhaler. The drug is delivered from a nasal solution as a fine mist or from a powder as an aerosol.

The term “subject” or “patient” refers to an animal, preferably a mammal, most preferably a human, who is the object of treatment, observation or experiment.

The term “therapeutically effective amount” means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The following non-limiting case examples are presented to further illustrate the present invention: In the following examples, unless otherwise stated, the ointment contained about 0.15-0.22 mg of topiramate per ml of ointment (Thermaderm, an aqueous vanishing cream emollient) and sodium bicarbonate in an amount to provide a pH of about 7.8-8.

Case 1. A young male presented with ezema particularly of his hands which were cracked and bleeding with a history of lesions of on his elbows and back of shoulders. Topirimate cream ointment was applied once a day and his hands cleared with resolution of the lesions after four days.

Case 2: An adolescent male presented with facial eczema in front of his left ear usually treated by corticosteroides which caused the lesions to recede and resolve only to return within weeks of cessation. Topiramate cream ointment was applied once a day with shrinkage and resolution of lesions after five days and then every other day for the past four months without the reoccurrence of symptoms.

Case 3: Poison Ivy—A young boy with known sensitivity to poison ivy presents with typical raised red vesicular lesions on his hands and left forearm. Topiramate cream/ointment was applied daily with the disappearance of lesions after 3 days.

Case 4: Poison Ivy—A young male having a history of extreme sensitivity to poison ivy presented with the entire right lower quadrant of his back covered with red, vesicular, and oozing lesions. These lesions cleared after four days of once a day application of Topiramate, at which time the application was stopped, leaving the skin dry with dead scales/scabs. The patient was seen four days later in this condition and began to apply Topirmate cream/lotion ever other day with complete resolution over the next six days.

Case 5: Contact dermatitis to jewelry. An adult male had a recurring (i.e. every 3 or 4 months) red oozing dermatitis underneath a 14 carat gold and stone ring on the third ring finger. This was usually treated with topical 0.1% mometasone furoate once daily for six or seven days with clearing of the lesion. Topiramate cream/ointment was begun with complete clearing in two days and no reoccurrence after three months.

Case 6. Contact dermatitis to jewelry. An adult female presented with a recurring red oozing dermatitis under a tight fitting silver/gold stone bracelet on her left wrist occurring especially during the winter and usually treated with topical corticosteroids over a five day period. Topiramate cream/ointment was applied daily with complete clearing in two days.

A petrolatum based ointment was used in all of the following cases 7-10 which contained petrolatum, mineral oil, ceresin wax and wool wax commercially available from Carolina Medical Products Co. POB 147, Farmville, N.C. 27828.

Case 7: An adult male presented with psoriatic minimally raised lesions in and on the inner surfaces of his ears, especially his right one which was worse in the winter, and treated with topical corticosteriods. Topiramate cream/ointment was begun once daily with clearing after five days.

Case 8: An adult male presented with mild psoriasis of his right elbow but with severe disfiguring raised hyperkeratotic psoriatic lesions of his right large toenail. This had been unresponsive to corticosteroids even when the disfigured toenail was cut back to apply corticosteroids to the nail bed. Topiramate cream/ointment was begun once daily to the exposed nail bed over a four month period with nail growing back to approximately 60% of normal with mildly raised striations. At that time a petrolatum base ointment with 0.45 mg of topiramate per milliliter was applied twice a day with a further 20% improvement after two weeks. The nail now had only minor surface irregularities but without the underlying hyperkeratotic nail bed.

### TISSUE DAMAGE

Case 9: An adult female presented with self-inflicted cuts and scratches to both forearms not requiring stitches but causing significant bleeding. Petrolatum base ointment mixed with 0.4 mg/ml topiramate was applied twice a day to one forearm and neosporin ointment to the other after both were cleaned with soap and water. Complete healing occurred in approximately 72 hours with the topiramate treated forearm while the neosporin treated arm did not heal until approximately 48 hours later.

Case 10: A right handed adult male presented directly after accidentally lifting a hot pot with both hands in a kitchen. He experienced more pain in the right hand than in the left. He was offered petrolatum based ointment mixed with 0.4 mg/ml topiramate for both hands but only accepted treatment for the right hand. The next day he returned to request ointment for his left hand since increasing pain with vesicle and blister formation consistent with a second degree burn had occurred, but none had occurred on the right hand.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

### References

1. Brazzini B.Pimpinalli N.: New and established topical cortisosteroids in dermatology: clinical pharmacology and therapeutic use, Ann J Clin Dermatol 2002: 3(1): 47-48

2. Baumann L. Francise K.: is Topical Glucocorticoids, chap. 243 in Fitzpatrick's Dermatology Fifth Edition IM. Freedberg, A. Z. Eisin, K. Woff et al eds 1999, p. 2713-2717.

3. Ashcroft DM, Li Wan PA, Griffiths CE. Therapeutic Strategies for Psoriasis J. Clin Pharm Ther 2000; 25 1-10.

4. Sauer's Manual of Skin Diseases J. C. Hall Ed. Chap. 5 Dernatologic Therapy Eighth Edition Lipponcott, Williams and Wilkins, New York, p. 33

5. AHFS Drug Information chap. 40:08 Alkalinizing Agents American Society of Health-System Pharmacists Bethesda, Md. 2002: p. 2497-2505

6. AHFS Drug Information chap. 56:64 Antacids and adsorbents. American Society of Health System Pharmaceuticals, Bethesda, Md. 2002, p. 2772-2777.

What is claimed is:

1. A method for treating a patient suffering from dermatoses or tissue damage or both which comprises topically applying a therapeutically effective amount for treating dermatitis or tissue damage or both a compound of the formula (I): 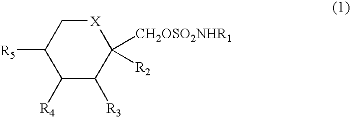
X is CH₂ or oxygen;
R₁ is hydrogen or alkyl and
R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II): 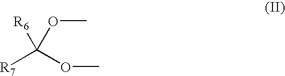
wherein
R₆ and R₇ are the same or different and are hydrogen, lower alkyl or are alkyl and are joined to form a cyclopentyl or cyclohexyl ring.

2. The method of claim 1 wherein said dermatoses and tissue damage are dermatoses and tissue damage, respectively that have shown responsiveness to the topical application of corticosteroids.

3. The method of claim 1 wherein said dermatoses is selected from the group consisting of Seborrheic dermatitis, Intertrigo, Primary irritant dermatitis, Papular Urticaria, Lichen simplex chronicus, Lupus erythematosus, Pemphigus, Lichen planus, Granuloma annulate, Necrobiosis lipodica diabeticorum, Sarcoidosis, insect bites, primary irritant dermatitis, contact dermatitis, allergic(acute phase) dermatitis, atopic dermatitis, vitiligo, eczema, parapsoriasis, psoriasis, skin burn, photosensitive dermatoses and generalized, winter, senile and essential pruritis.

4. The method of claim 1 wherein said compound is topiramate.

5. The method of claim 1 wherein the dermatoses is from poison ivy, poison oak, psoriasis, eczema or jewelry.

6. The method of claim 1 wherein the dermatoses is from poison ivy.

7. The method of claim 1 wherein the dermatoses is from psoriasis.

8. The method of claim 1 wherein the dermatoses is from eczema.

9. The method of claim 1 wherein the dermatoses is from jewelry.

10. The method of claim 1 wherein the compound is employed in a composition that is in the form of an aerosol, foam, medicated tape, skin patch, paste, oil, tincture, aqueous solution, cream, or ointment.

11. The method of claim 10 wherein the composition has pH of about 7-10.

12. The method of claim 10 wherein the composition has pH of about 7-8.

13. A method for treating a patient suffering from dermatoses or tissue damage or both other than psoriasis which comprises administering to said patient a therapeutically effective amount for treating said dermatoses or tissue damage or both a compound of the formula (I): 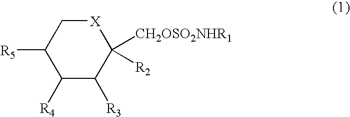
X is CH₂ or oxygen;
R₁ is hydrogen or alkyl and
R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II): 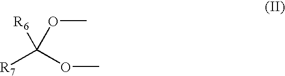
wherein
R₆ and R₇ are the same or different and are hydrogen, lower alkyl or are alkyl and are joined to form a cyclopentyl or cyclohexyl ring.

14. The method of claim 13 wherein said dermatoses is selected from the group consisting of Seborrheic dermatitis, Intertrigo, Primary irritant dermatitis, Papular Urticaria, Lichen simplex chronicus, Lupus erythematosus, Pemphigus, Lichen planus, Granuloma annulate, Necrobiosis lipodica diabeticorum, Sarcoidosis, insect bites, primary irritant dermatitis, contact dermatitis, allergic(acute phase) dermatitis, atopic dermatitis, vitiligo, eczema, parapsoriasis, skin burn, photosensitive dermatoses and generalized, winter, senile and essential pruritis.

15. The method of claim 13 wherein the dermatoses is from poison ivy.

16. The method of claim 13 wherein the dermatoses is from eczema.

17. The method of claim 13 wherein the dermatoses is from jewelry.

18. The method of claim 13 wherein and compound is topiramate.
